(19) United States
(12) Patent Application Publication
(10) Pub. No.: US 2025/0283099 A1
(43) Pub. Date: Sep. 11, 2025

(54) GENETICALLY ENGINEERED PLANT OR PART THEREOF ADAPTED FOR THE PRODUCTION OF RECOMBINANT PROTEINS AND PROCESS THEREOF

(71) Applicant: CORE BIOGENESIS, EVRY (FR)

(72) Inventor: Chouaib MEZIADI, EVRY (FR)

(21) Appl. No.: 18/282,080

(22) PCT Filed: Mar. 10, 2022
(86) PCT No.: PCT/EP2022/056113

§ 371 Date: Sep. 14, 2023

(30) Foreign Application Priority Data
Mar. 16, 2021 (EP) 21305319.2

(51) Int. Cl. C12N 15/82; A01H 1/00; A01H 1/04

(52) U.S. Cl. CPC C12N 15/8218; A01H 1/045; A01H 1/108

(57) ABSTRACT

A genetically engineered plant or part thereof adapted for the production of recombinant proteins, the plant comprising at least one inactivated gene involved in the Transcriptional Gene Silencing (TGS) and/or Post Transcriptional Gene Silencing (PTGS) mechanism and at least one nucleic acid construct comprising a viral promoter coupled to a nucleic acid construct comprising a coding sequence of a recombinant protein. A nucleic acid construct, a process for producing a recombinant protein, a method for obtaining a genetically engineered plant or part thereof and a fusion protein comprising a recombinant protein and an oil-body protein produced by, or obtained from the genetically engineered plant or new-grown plants from the seeds of the genetically engineered plant.

### DESCRIPTION OF THE TEXT FILE SUBMITTED ELECTRONICALLY

The sequence listing associated with this application is provided in text format in lieu of a paper copy, and is hereby incorporated by reference into the specification. The name of the text file containing the Sequence Listing is “072797 EP Sequences_ST25.txt”.

### FIELD OF THE INVENTION

The present invention relates to a genetically engineered plant or part thereof adapted for the production of recombinant proteins, the plant comprising at least one inactivated gene involved in the Transcriptional Gene Silencing (TGS) and/or Post Transcriptional Gene Silencing (PTGS) mechanism and at least one nucleic acid construct comprising a strong promoter coupled to a nucleic acid construct comprising a coding sequence of a recombinant protein.

The present invention also relates to a nucleic acid construct, a process for producing a recombinant protein, a method for obtaining a genetically engineered plant or part thereof and a fusion protein comprising a recombinant protein and an oil-body protein produced by, or obtained from the genetically engineered plant or new-grown plants from the seeds of the genetically engineered plant.

### BACKGROUND PRIOR ART

A recombinant protein is a protein produced by cells whose DNA has been modified by genetic engineering. Recombinant proteins can be molecules of pharmaceutical, therapeutic, cosmetic interest, molecules used in research and development or in the field of agroindustry. So as to be produced in large quantities, the host cell whose DNA has been modified must grow in large quantities and the protein produced must be functional and easy to purify. In particular, the recombinant protein coding sequence must be implemented in the host cell in a large number of copies with a promoter which allows an important rate of transcription or which can be induced.

Up to now, available molecules are mainly produced in bacteria, mammalian cells and, to a lesser extent, in yeasts and insect cells. These conventional cell culture-based systems are useful but limited by high cost, low efficiency or low yield. Besides, mammalian cells as production hosts raise safety and ethical concerns. Therefore, global production capacities for recombinant proteins are currently insufficient or see their field of applications limited.

Plants as recombinant protein production hosts have been considered as a viable option. Indeed, they provide a valuable alternative to fermentation-based systems for the production of recombinant proteins, having numerous advantages. First, they have the ability to perform post-translational modifications such as correct folding, glycosylation and disulfide bonds that are essential for most mammalian proteins' activity. Particularly, such post-translational modifications are essential for proteins to be effective. Without such post-translational modifications, recombinant proteins obtained may not be functional and artificially reproducing these post-translational modifications may be considered impossible at the moment or too costly. Secondly, there is a greatly reduced risk of contamination with human pathogens, which is a serious concern with mammalian cells in bioreactors. Thirdly, plant growth requirements are simple and not expensive compared to traditional fermentation-based systems, allowing for low-cost scale-up. Whole plants have quite simple growth needs compared to mammalian cell cultures as they only need light as an energy source and minerals to grow. In addition, transient expression systems are able to produce proteins including vaccine candidates in high quantity within a short time period (4-6 weeks), an efficient time scale in case of pandemics or flu epidemics (Gleba Y, Klimyuk V, Marillonnet S. Magnifection—a new platform for expressing recombinant vaccines in plants. Vaccine. 2005 Mar 18;23(17-18):2042-8. doi: 10.1016/j.vaccine.2005.01.006. PMID: 15755568; Musiychuk, Konstantin et al. “A launch vector for the production of vaccine antigens in plants.” Influenza and other respiratory viruses vol. 1, 1 (2007): 19-25. doi: 10.1111/j.1750-2659.2006.00005.x).

To obtain genetically engineered plants, solutions such as stable nuclear transformation may be used, which is a flexible solution with efficient scale-up. Plastid genome engineering may also be used as their highly polyploid and circular DNA allows a large number of transgene copies. However, plastidial expression is prokaryotic in nature and cannot do post-translational modifications described earlier. Transient expression using agroinfiltration also allows production of a recombinant protein with a maximum yield between 2 and 5 days post infiltration.

The main drawback of the stable nuclear transformation system is low expression levels which allows between 0.001% and 1% of TSP (Total Soluble Proteins) mainly due to gene silencing by Transcriptional Gene Silencing (TGS) and posttranscriptional gene silencing (PTGS). These production rates are far from being enough to compete with traditional fermentation-based systems. Low levels of protein accumulation also increase the cost of purification to non-economic levels. For agroinfiltration, in addition to the need of setting up a complex and costly system to produce on a large scale, transgene RNAs are frequently recognized by plant cells as being “foreign” and silenced by post-transcriptional gene silencing (PTGS). Even if this PTGS can be overcome in some cases by co-expressing a viral suppressor of gene silencing, such as the p19 protein of tomato bushy stunt virus (TBSV), the production in agroinfiltrated leaves remains between 1% TSP and 15% TSP. Further, this method is time-consuming, its implementation requires expert manipulations, and the extraction and purification process is as expensive as conventional systems.

One of the major criteria for high-level expression of a protein is the use of a strong promoter. Viral promoters are by far the most used ones. However, the stronger the expression, the more irregularity is observed. This is mainly due to a defense mechanism called gene silencing triggered against the expression of these transgenes which are treated like invasive nucleic acids due to their high level expression under the strong promoter. Gene silencing can be activated against the introduced gene or against its transcripts by the transcriptional or post-transcriptional gene silencing (PTGS and TGS) respectively. This inactivation corresponds to the specific degradation of messenger RNA (mRNA) encoded by the transgene and the production of double-stranded RNAs (dsRNA) by RNA-DEPENDENT RNA POLYMERASES (RDRs). Then, dsRNAs are processed into 21 to 24 nt (nucleotides) duplexes according to the DICER-LIKE (DCL) enzyme and are protected from exonucleases through 2′O-methylation by HUA ENHANCER 1 (HEN1). Finally, one strand of the small RNA duplexes is loaded into ARGONAUTE (AGO) proteins therefore forming the RNA-inducing silencing complex (RISC) which will drive sequence-specific mRNA cleavage & translational repression or RNA directed DNA methylation (RdDM) (Martínez de Alba AE, Elvira-Matelot E, Vaucheret H. Gene silencing in plants: a diversity of pathways. Biochim Biophys Acta. 2013 Dec; 1829(12):1300-8. doi: 10.1016/j.bbagrm.2013.10.005. Epub 2013 Nov 1. PMID: 24185199).

Several strategies can be adopted to avoid this phenomenon, for example by expressing simultaneously a silencing suppressor in addition to the gene of interest. These suppressors generally have a viral origin. For example p19 protein from the Tomato Bushy Stunt Virus (TBSV) is a suppressor which can be co-expressed with a recombinant protein which results in a highly increased expression level of the protein of interest (Voinnet et al. Suppression of gene silencing: a general strategy used by diverse DNA and RNA viruses of plants. Proc Natl Acad Sci USA. 1999). However, such suppressors of gene silencing can affect the development of the plant and exhibit heterogeneity when they are co-infiltrated in transient expression.

Ultimately, the cost of the purification process with plants could be identical to the one used in fermentation-based systems and onerous steps of extraction-purification of the recombinant protein from plant tissue are required. Often, the recombinant protein is inserted into the matrix of plant tissue, which makes its extraction and purification difficult to carry out, even though this step must be carried out as quickly as possible due to the risk of proteolysis in plant extracts. In conventional systems, recombinant proteins are extracted along with many other compounds such as nucleic acids, chlorophyll, alkaloids, phenolics, polysaccharides and proteases. The problem is that these compounds can not only bind and degrade the recombinant protein but also foul chromatography resins and crossflow membranes resulting in reducing final product quality and yield or adding to manufacturing costs.

Therefore, global production capacities for recombinant proteins are currently insufficient or see their field of applications limited. Hence there is a need to find alternative solutions and/or innovative technologies.

The invention seeks to overcome the aforementioned drawbacks of the prior art as it aims to provide a genetically engineered plant which allows producing large quantities of recombinant proteins which are functional, can be easily extracted and purified, and which may be used for multiple applications.

### SUMMARY OF THE INVENTION

To this effect, the present invention relates to a genetically engineered plant or part thereof adapted for the production of recombinant proteins comprising:
at least one inactivated gene involved in the Transcriptional Gene Silencing (TGS) and/or Post Transcriptional Gene Silencing (PTGS) mechanism corresponding to:
   (1) at least one knocked-out gene involved in the TGS and/or PTGS mechanism; and/or
   (2) at least one nucleic acid RNA interference construct targeting the RNA molecules expressed from the at least one gene involved in the TGS and/or PTGS mechanism;
at least one nucleic acid construct comprising a strong promoter coupled to a polynucleotide sequence comprising a coding sequence of a recombinant protein;
wherein (i) the coding sequence of the recombinant protein is linked in a reading frame to an oil body protein coding sequence; and/or (ii) the at least one inactivated gene involved in the TGS and/or PTGS mechanism includes at least one inactivated gene involved in the TGS mechanism and at least one inactivated gene involved in the PTGS mechanism.

By “at least one inactivated gene” as used herein it is understood a gene which cannot encode an RNA or a protein or encodes no functional protein, or which encodes a protein with reduced activity by at least 70%, preferably at least 90%. Said “inactivated gene” can encode a protein with reduced functionality, or be a fully inactivated gene, encoding no functional protein or not encoding any protein. An inactivated gene involved in the TGS or PTGS mechanism in the present invention is usually a wild-type gene, i.e. a wild-type gene which, due to the genetically engineered inactivation, cannot encode an RNA or protein, or encodes no functional protein, or encodes a protein with reduced activity, by at least 70%, preferably at least 90%.

By “gene involved in the Transcriptional Gene Silencing (TGS) and/or Post Transcriptional Gene Silencing (PTGS) mechanism”, it is particularly understood as any gene involved in the TGS and/or PTGS mechanism which would prevent the transcription or the RNA product of the genetically engineered at least one nucleic acid construct, which comprises a strong promoter coupled to a polynucleotide sequence comprising a coding sequence of a recombinant protein, wherein such coding sequence of a recombinant protein may be linked in a reading frame to an oil body protein coding sequence.

By “knocked-out gene”, it is understood a gene which has been genetically engineered and which has at least one genetic alteration in its coding sequence which leads to the inability to encode an RNA or a protein or which leads to a protein which is not functional, or with a reduced activity, by at least 70%. By genetic alteration, it is intended to signify at least one among the following modifications: an insertion of a stop codon, an insertion of a nucleotide resulting in the creation of a stop codon, a shift of the nucleotide reading frame resulting in a protein which is not functional, the introduction of a recombination site, such as Cre-Lox, or any modification which has a highly suppressive effect of at least 70%, preferably at least 90% on the expression of the target gene. Other modifications altering the coding sequence of the gene so as to knock it out may also be used. Such genetic alterations typically result from the use of a method selected among CRISPR-Cas 9, synthetic RNAi, TALENs, Meganuclease, TILLING and Zinc finger nuclease but other methods may be suitable. As an alternative, other methods such as physical and chemical mutagenesis by Gamma irradiation or EMS treatment may be used. Preferably, the method used for generating the genetic alterations is CRISPR-Cas9.

By “at least one nucleic acid RNA interference construct targeting the RNA molecules expressed from the at least one gene involved in the TGS and/or PTGS mechanism”, it is understood that the at least one gene involved in the TGS and/or PTGS mechanism allows generation of RNAi duplex, generated from the natural TGS and PTGS mechanism or by a hairpin artificial structure.

By “at least one nucleic acid construct comprising a strong promoter coupled to a polynucleotide sequence comprising a coding sequence of a recombinant protein”, it is intended to signify a genetically engineered construct which has been inserted into the plant so as to enable the plant to produce the recombinant protein. The said ‘at least one nucleic acid construct’ may also be referred to as a transgene. By “at least one nucleic acid construct comprising a strong promoter coupled to a polynucleotide sequence comprising a coding sequence of a recombinant protein linked in a reading frame to an oil body protein coding sequence”, it is intended to signify a genetically engineered construct which has been inserted into the plant so as to enable the plant to produce the recombinant protein and the oil body protein.

By “oil body protein”, it is understood as a protein which may be part of a lipid-containing structure which is typically intracellular. Typically, a lipid-containing structure occurring in plants comprising triacylglycerol molecules or other neutral lipids surrounded by a phospholipid half-unit membrane and oil body proteins.

Regarding oil-body proteins, the water insoluble oil fraction is stored in discrete subcellular structures variously known in the art as oil bodies which may be referred as oleosomes, lipid bodies, lipid droplets or spherosomes. Besides a mixture of oils, such as triacylglycerides or other neutral lipids, which chemically are defined as glycerol or fatty alcohol esters of fatty acids, oil bodies comprise phospholipids and a number of associated proteins, collectively termed oil body proteins.

In the present invention “a strong promoter” is used. It will be understood that the strength of a promoter is determined in part by the attributes of the cell in which it operates. By “a strong promoter”, it is intended to mean a promoter which results in high-level expression of gene elements, which causes mRNAs to be initiated at high frequencies. The term “strong promoter” is a term well known and widely used in the art and many strong promoters are known in the art, or can be identified by routine experimentation. It may be a native promoter or a promoter which is not native of the engineered plant, such as another plant promoter, a viral promoter, a bacterial promoter or even a synthetic promoter. Each of these promoters may be used in any embodiment disclosed herein. Preferably, the strong promoter is a viral promoter. By “a viral promoter”, “a bacterial promoter”, “a synthetic promoter”, it is intended to mean any viral, bacterial, synthetic promoter which may be used in plants.

For instance, as viral promoters, the promoter from CMV (P CMV) the mtHP promoter (Xiao, K., Zhang, C., Harrison, M., & Wang, Z. (2004). Isolation and characterization of a novel plant promoter that directs strong constitutive expression of transgenes in plants. Molecular Breeding, 15, 221-231; Patent EP1608761B1), the MMV FLt promoter (Dey, N., Maiti, I.B. Structure and promoter/leader deletion analysis of mirabilis mosaic virus (MMV) full-length transcript promoter in transgenic plants. Plant Mol. Biol. 40, 771-782 (1999); U.S. Pat. No. 6,420,547B1), the rbcS1 promoter (Outchkourov NS, Peters J, de Jong J, Rademakers W, Jongsma MA. The promoter-terminator of chrysanthemum rbcS1 directs very high expression levels in plants. Planta. 2003 Apr) and the Chlorella virus gene promoter (Mitra A, Higgins DW, Rohe NJ. A Chlorella virus gene promoter functions as a strong promoter both in plants and bacteria. Biochem Biophys Res Commun. 1994 Oct 14) may be used. Also, the 35 S viral promoter may be used, for instance CaMV 35S or its derivatives including the tandem sequence. Also double 35S CaMV or CSMV promoter may be used.

Advantageously, the at least one inactivated gene involved in the TGS or PTGS mechanism is chosen among the Dicer-like (DCL) family, the Suppressor of Gene Silencing (SGS) family, the Argonaute (AGO) family, the RNA dependent RNA polymerase (RdR) family and combinations thereof.

Advantageously, the at least one inactivated gene involved in the TGS or PTGS mechanism includes at least one inactivated gene involved in the TGS mechanism and at least one inactivated gene involved in the PTGS mechanism. For instance, the at least one gene involved in the TGS mechanism may be RDR2 or DCL3 or one of AGOs genes. For instance the at least one gene involved in the PTGS mechanism may be RDR6 or DCL2 or DCL4.

Advantageously, the at least one inactivated gene involved in the TGS and/or PTGS mechanism includes the RdR2 gene and the RdR6 gene.

Advantageously, the oil body protein coding sequence is the coding sequence of an oil body protein selected among oleosin, caleosin, steroleosin, perilipin or caveolin.

The coding sequence of the recombinant protein in the at least one nucleic acid construct is not limited. The coding sequence of the recombinant protein may be related to proteins used in cosmetic industry, pharmaceutical industry, food industry or agriculture. Advantageously, the coding sequence of the recombinant protein may be selected among the coding sequence of antibodies, growth factors, enzymes, structural proteins, receptors and signaling proteins and antigens.

Advantageously, the coding sequence of a recombinant protein is the coding sequence or a partial coding sequence of a protein or epitope of a virus. Preferably, such virus is a virus causing human disease. Even more preferably, such protein or epitope of a virus is a protein or epitope of the SARS-COV-2 virus, for instance the peplomers or Spike protein of SARS-COV-2.

Advantageously, the plant is a genetically engineered oleaginous plant or part thereof, preferably selected among the following genus: Arachis, Camelina Theobroma, Brassica, Gossypium, Olea, Ricinus, Glycine, Helianthus, Elaeis, Carthamus and Linum.

Alternatively, the plant is a genetically engineered Nicotiana spp including Nicotiana benthamiana or Camelina sativa or part thereof. A genetically engineered plant being a double mutant RdR2 RdR6 of the Nicotiana benthamiana and Camelina sativa species is particularly adapted for the expression of the nucleic acid construct comprising a strong promoter coupled to a polynucleotide sequence comprising a coding sequence of a recombinant protein in reading frame to an oil body protein coding sequence.

Advantageously, the part thereof is at least one part selected among: the seeds, the pollens, the flowers, the roots, the stems, the spores, the mega- or micro-spores, embryos and the vegetative organs of non-flowering plants.

The present invention also relates to a nucleic acid construct comprising a strong promoter coupled to a polynucleotide sequence comprising a coding sequence of a recombinant protein linked in a reading frame to an oil body protein coding sequence.

Advantageously, the oil body protein coding sequence is a coding sequence of oleosin, caleosin, steroleosin, perilipin or caveolin.

Advantageously, the strong promoter of the nucleic acid construct may be a viral promoter such as described previously.

The coding sequence of the recombinant protein in the at least one nucleic acid construct is not limited. The coding sequence of the recombinant protein may be related to proteins used in cosmetic industry, pharmaceutical industry, food industry or agriculture. Advantageously, the coding sequence of the recombinant protein is selected among the coding sequence of antibodies, growth factors, enzymes, structural proteins, receptors and signaling proteins and antigens.

The present invention also relates to a process for producing a recombinant protein in a genetically engineered plant or part thereof, comprising the steps of:
inactivating at least one gene involved in the TGS and/or PTGS mechanism in a plant or part thereof by:
   (1) generating at least one genetic alteration in the coding sequence of the at least one gene involved in the TGS and/or PTGS mechanism,
      such generating step being performed by using a method selected among: CRISPR-Cas 9, TALENs, Meganuclease, TILLING, Zinc finger nuclease, synthetic RNAi, physical or chemical mutagenesis and combinations thereof; and/or
   (2) inserting in the plant at least one nucleic acid RNA interference construct targeting the RNA molecules expressed from the at least one gene involved in the TGS and/or PTGS mechanism;
inserting in the plant at least one nucleic acid construct comprising a strong promoter coupled to a polynucleotide sequence comprising a coding sequence of a recombinant protein,
   wherein (i) the coding sequence of the recombinant protein is linked in a reading frame to an oil body protein coding sequence; and/or (ii) the step of inactivating at least one gene includes the inactivation of at least one gene involved in the TGS mechanism and at least one gene involved in the PTGS mechanism,
   wherein the step of inserting is performed by using a method selected among: CRISPR-Cas 9, TALENs, Meganuclease, Zinc finger nuclease with particle gun transformation and Agrobacterium tumefaciens,
   wherein the step of inactivating may be performed after the step of inserting, and
optionally recovering the recombinant protein and the oil body protein and optionally purifying the recombinant protein recovered.

Preferably, the method used for generating the genetic alterations is CRISPR-Cas9.

Advantageously in that process, the at least one inactivated gene involved in the TGS or PTGS mechanism is chosen among the Dicer-like (DCL) family, the Suppressor of Gene Silencing (SGS) family, the Argonaute (AGO) family the RNA dependent RNA polymerase (RdR) family and combinations thereof.

Advantageously, the at least one inactivated gene involved in the TGS or PTGS mechanism includes at least one inactivated gene involved in the TGS mechanism and at least one inactivated gene involved in the PTGS mechanism. For instance, the at least one gene involved in the TGS mechanism may be RDR2 or DCL3 or one of AGOs genes. For instance the at least one gene involved in the PTGS mechanism may be RDR6 or DCL2 or DCL4.

Advantageously in that process, the at least one inactivated gene involved in the TGS and/or PTGS mechanism includes the RdR2 gene and the RdR6 gene.

Advantageously in that process, the oil body protein coding sequence is a coding sequence of oleosin, caleosin, steroleosin, perilipin or caveolin.

The coding sequence of the recombinant protein in the at least one nucleic acid construct is not limited. The coding sequence of the recombinant protein may be related to proteins used in cosmetic industry, pharmaceutical industry, food industry or agriculture. Advantageously in that process, the coding sequence of the recombinant protein is selected among the coding sequence of antibodies, growth factors, enzymes, structural proteins, receptors and signaling proteins and antigens.

Advantageously in that process, the coding sequence of a recombinant protein is the coding sequence or a partial coding sequence of a protein or epitope of a virus. Preferably, such virus is a virus causing human disease. Even more preferably, such protein or epitope of a virus is a protein or epitope of the SARS-COV-2 virus, for instance the peplomers or Spike protein of SARS-COV-2

Advantageously in that process, the plant is a genetically engineered oleaginous plant or part thereof, preferably selected among the following genus: Arachis, Camelina, Theobroma, Brassica, Gossypium, Olea, Ricinus, Glycine, Helianthus, Elaeis, Carthamus and Linum.

Alternatively in that process, the plant is a genetically engineered Nicotiana spp including Nicotiana benthamiana or Camelina sativa or part thereof.

Advantageously in that process, the part thereof is at least one part selected among: the seeds, the pollens, the flowers, the roots, the stems, the spores, mega- or micro-spores, the embryos and the vegetative organs of non-flowering plants.

The present invention also relates to a method for obtaining a genetically engineered plant or part thereof as described above, the method comprising:
inactivating at least one gene involved in the TGS and/or PTGS mechanism gene in a plant or part thereof by:
   (1) generating at least one genetic alteration in the coding sequence of one or more gene involved in the TGS and/or PTGS mechanism, such generating step being performed by using a method selected among: CRISPR-Cas 9, TALENs, Meganuclease, TILLING, Zinc finger nuclease, synthetic RNAi, physical or chemical mutagenesis and combinations thereof; and/or
   (2) implementing RNA interference genes targeting the RNA molecules expressed from the TGS and/or PTGS mechanism genes,
      and
inserting in the plant a nucleic acid construct comprising a strong promoter coupled to a nucleic acid sequence comprising a coding sequence of a recombinant,
   wherein (i) the coding sequence of the recombinant protein is linked in a reading frame to an oil body protein coding sequence; and/or (ii) the step of inactivating at least one gene includes the inactivation of at least one gene involved in the TGS mechanism and at least one gene involved in the PTGS mechanism,
   such step of inserting being performed by using a method selected among: CRISPR-Cas 9, TALENs, Meganuclease, Zinc finger nuclease with particle gun transformation and Agrobacterium tumefaciens,
   wherein the inactivation step can be performed after the step of inserting.

Advantageously, the method further comprises:
collecting the seeds of the genetically engineered plant with at least one inactivated gene involved in the TGS and/or PTGS mechanism and the nucleic acid construct inserted;
growing new plants from the seeds and/or plant tissues; and
selecting the heterozygotes or homozygotes from new-grown plants which have at least one inactivated gene involved in the TGS and/or PTGS mechanism and the nucleic acid construct, and all progeny and genetic crosses using these plants.

The present invention also relates to a fusion protein comprising a recombinant protein and an oil-body protein produced by, or obtained from:
the genetically engineered plant or part thereof as described above, or
the genetically engineered plant or part thereof obtained from the method as described above, or
the heterozygotes or homozygotes new-grown plants selected as described above.

The present invention also relates to a seed from the genetically engineered plant or part thereof as described above or recovered or purified from the process as described above.

The present invention also relates to a callus of the genetically engineered plant of the invention.

Although it is not admitted that an essentially biological process may allow obtaining a genetically engineered plant or part thereof of the present invention, or a nucleic acid construct of the present invention, or a fusion protein comprising a recombinant protein and an oil-body protein produced by, or obtained from the genetically engineered plant or part thereof of the present invention or obtained from the heterozygotes or homozygotes new-grown plants selected of the present invention, it is stated that the claimed products are obtained by means which are not essentially biological process and the claimed methods do not correspond to essentially biological process.

Further characteristics, details and advantages of the invention will emerge from the description made with reference to the annexed drawings given by way of example.

### BRIEF DESCRIPTION OF THE DRAWINGS

The invention will be better understood and its various characteristics and advantages will emerge from the following description of a number of exemplary embodiments and its appended figures in which:

FIG. 1 displays a first diagram representing the TGS and PTGS mechanisms;

FIG. 2 displays a second diagram representing the strategy used according to a particular embodiment of the present invention.

### DETAILED DESCRIPTION OF THE INVENTION

In this specification, the invention will be described by way of examples. However, the invention is not restricted to these examples.

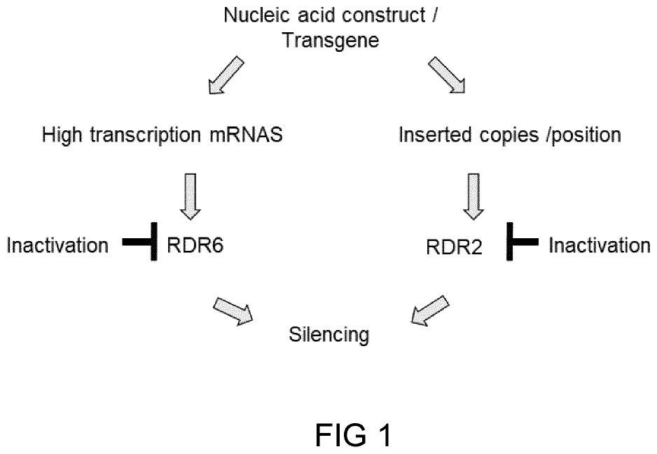

FIG. 1 displays a first diagram representing the TGS and PTGS mechanisms. In a classic functioning cell, a double strand DNA (dsDNA) is transcribed by RNA polymerase II in single strand RNAs (ssRNA). Such ssRNAs are then replicated by RdR6 to double strand RNAs (dsRNAs) which mediates the production of 21-22 nucleotides by DCL4 or 2 or 24 nucleotides by DCL3. HEN1 allows the methylation of such nucleotides which are then used as guides by the AGOs proteins for the silencing process. These proteins are components of the RNA-Induced Silencing Complex (RISC) which allows gene silencing, and according to RdR6, the post-transcriptional gene silencing (PTGS) mechanism.

On the other side of FIG. 1 is shown the similar transcriptional gene silencing (TGS) mechanism which is related to RdR2 and DCL3.

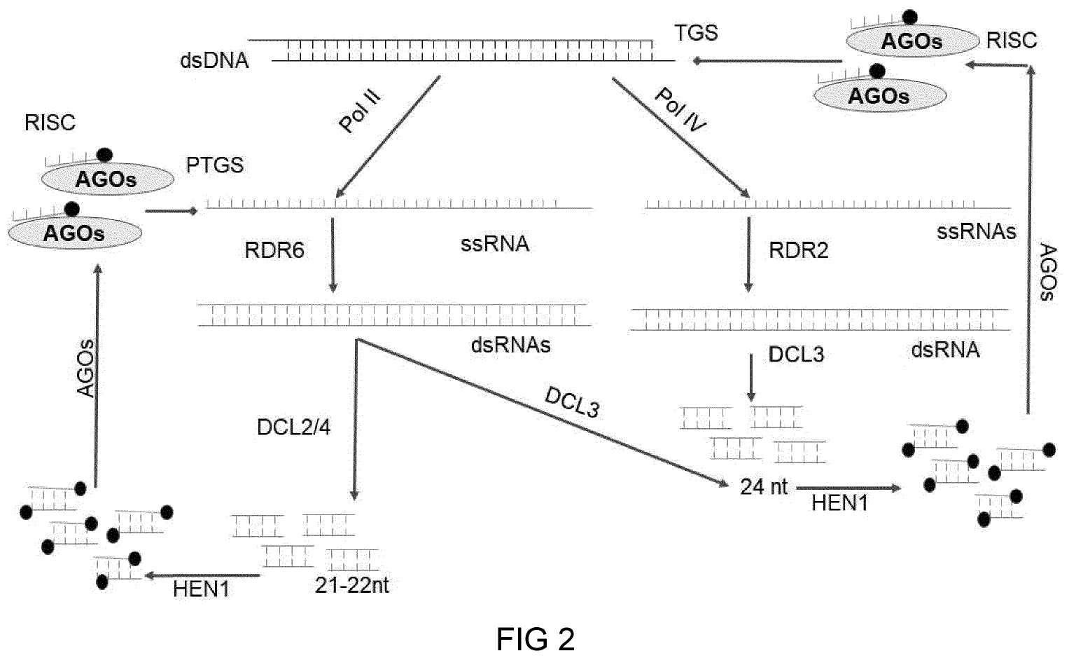

FIG. 2 displays a second diagram representing the strategy used according to a particular embodiment of the present invention. In FIG. 2, the nucleic acid construct, or transgene, which has been inserted in the plant, allows the high transcription of mRNAs as the strong promoter of the nucleic acid construct is coupled to a polynucleotide sequence of a recombinant protein. Moreover, the nucleic acid construct is inserted in multiple copies, at multiple positions. In a plant which has only been engineered by the insertion of the nucleic acid construct, RdR6 would allow the silencing at post transcription level while RdR2 would allow the silencing at transcription level, resulting in the protection of the plant against the expression of the nucleic acid construct.

According to a particular embodiment of the invention, the RdR2 and RdR6 genes are inactivated by the generation of at least one genetic alteration in the coding sequence of the RdR2 and RdR6 genes of the plant cells. Such inactivation prevents the silencing mechanism to be effective against the transcription of the nucleic acid construct inserted. Therefore, the plant cells are used as a production unit so as to obtain the recombinant protein of the recombinant protein coding sequence comprised in the nucleic acid construct.

According to present invention, the nucleic acid construct may comprise coding sequences of the recombinant protein and the oil body protein in a reading frame, allowing the expression of both coding sequences so as to obtain fusion proteins. By “linked in a reading frame” it is understood that the recombinant protein and the oil body protein coding sequences are set on consecutive triplets of DNA, which are non-overlapping. The order coding sequences of the recombinant protein and the oil-body protein is not relevant and they may be inverted.

The fusion protein may correspond to a tandem fusion, with the proteins being connected end-to-end or by protein linkers. Specific protein linkers may be particularly suitable for the purification process and methods of the present invention so as for these protein linkers to be specifically targeted so as to separate the recombinant protein from the oil-body protein. Such protein linkers may correspond to multiple glycine residues or proline residues but other protein linkers may be suitable.

Further, the fusion proteins may accumulate in oil bodies, which may be referred as oleosomes, containing mainly the recombinant protein and the oil body protein, such as oleosin. Other oil body proteins may be used, such as caleosin, steroleosin, perilipin and caveolin. Particularly, the oil bodies are present in the seeds and the leaves.

Among the strong promoters of the nucleic acid construct cited above, viral promoters may be used. For instance, as viral promoters, the promoter from CMV (P CMV) the mtHP promoter (Xiao, K., Zhang, C., Harrison, M., & Wang, Z. (2004). Isolation and characterization of a novel plant promoter that directs strong constitutive expression of transgenes in plants. Molecular Breeding, 15, 221-231; Patent EP1608761B1), the MMV FLt promoter (Dey, N., Maiti, I.B. Structure and promoter/leader deletion analysis of mirabilis mosaic virus (MMV) full-length transcript promoter in transgenic plants. Plant Mol. Biol. 40, 771-782 (1999); U.S. Pat. No. 6,420,547B1), the rbcS1 promoter (Outchkourov NS, Peters J, de Jong J, Rademakers W, Jongsma MA. The promoter-terminator of chrysanthemum rbcS1 directs very high expression levels in plants. Planta. 2003 Apr) and the Chlorella virus gene promoter (Mitra A, Higgins DW, Rohe NJ. A Chlorella virus gene promoter functions as a strong promoter both in plants and bacteria. Biochem. Biophys. Res Commun. 1994 Oct 14) may be used. Also, the 35 S viral promoter may be used, for instance CaMV 35S or its derivatives including the tandem sequence. Also double 35S CaMV or CSMV promoter may be used.

In the genetically engineered plant or part thereof of the present invention, or the associated process and methods, the inactivation of at least one gene involved in the Transcriptional Gene Silencing (TGS) and/or Post Transcriptional Gene Silencing (PTGS) mechanism allows blocking the triggering of silencing against the expression of the at least one nucleic acid which would be considered as invasive nucleic acids due to an high level of expression under a strong promoter such as for instance a viral promoter. Said inactivation prevents the specific degradation of messenger RNA encoded by the nucleic acid construct and/or prevents the production of double stranded RNAs (dsRNA) encoded by the nucleic acid construct. The inactivation may inhibit sequence-specific mRNA cleavage and translational repression or RNA directed DNA methylation.

The inactivation of the said at least one gene involved in the Transcriptional Gene Silencing (TGS) and/or Post Transcriptional Gene Silencing (PTGS) mechanism is not lethal to the genetically engineered plant.

In the present invention the at least one inactivated gene involved in the TGS or PTGS mechanism may be chosen among the Dicer-like (DCL) family, the Suppressor of Gene Silencing (SGS) family, the Argonaute family (AGO), the RNA dependent RNA polymerase (RdR) family and combinations thereof. However, the at least one gene to inactivate may vary depending on the species or genus of plants which is genetically engineered.

In the case of a genetically engineered plant comprising at least one nucleic acid construct comprising a strong promoter coupled to a polynucleotide sequence comprising a coding sequence of a recombinant protein linked in a reading frame to an oil body protein coding sequence, such a genetically engineered plant allows increasing the yield and simplifying the first steps of purification extraction by addressing the recombinant proteins to oil-bodies particularly present in leaves and seeds. Therefore, cell fractionation and purification of lipid bodies carrying the recombinant proteins by flotation gradient may be extracted from the genetically engineered plant or parts thereof and particularly from the leaves or seeds.

According to the alternatives of the present invention, on one hand, with RNAi inhibition modification the genetically engineered plant may be cultivated so as to be more resistant to pathogens. On the other hand, with knocked-out genes, the recombinant protein production yield is greater.

In a particular embodiment, the at least one inactivated gene involved in the TGS or PTGS mechanism includes at least one inactivated gene involved in the TGS mechanism and at least one inactivated gene involved in the PTGS mechanism. For instance, the at least one gene involved in the TGS mechanism may be RDR2 or DCL3 or one of AGOs genes. For instance the at least one gene involved in the PTGS mechanism may be RDR6 or DCL2 or DCL4.

Advantageously, the at least one inactivated gene involved in the TGS and/or PTGS mechanism includes the RdR2 gene and the RdR6 gene. Such genetically engineered plant or part thereof may be referred as a double mutant RdR2 RdR6, which increases the expression of the nucleic acid construct by disrupting homogeneously the silencing mechanism at the transcriptional and post-transcriptional level and without having an impact on plant development. A specific RdR2 RdR6 double mutant which is preferred is a RdR2 RdR6 double mutant of Nicotiana spp including Nicotiana benthamiana or Camelina sativa.

The coding sequence of the recombinant protein in the at least one nucleic acid construct is not limited. The coding sequence of the recombinant protein may be related to proteins used in cosmetic industry, pharmaceutical industry, food industry or agriculture. Advantageously, the coding sequence of the recombinant protein is selected among the coding sequence of antibodies, growth factors, enzymes, structural proteins, receptors and signaling proteins and antigens.

Advantageously, the coding sequence of a recombinant protein is the coding sequence of a protein or a partial coding sequence of a protein or epitope of a virus. Preferably, such virus is a virus causing human disease. Even more preferably, such protein or epitope of a virus is a protein or epitope of the SARS-COV-2 virus, for instance the peplomers or Spike protein of SARS-COV-2

By “optionally recovering the recombinant protein and the oil body protein”, it is understood that as soon as a genetically engineered plant or part thereof as indicated above is obtained, the genetically engineered plant or part thereof is able to express the coding sequence of the recombinant protein and the oil body protein by initiating the expression from the strong promoter. Due to a high expression activity caused by the strong promoter, the proteins which will be obtained will be in high quantity and they will gather in oil bodies, particularly in the seeds and in the leaves.

Further when using this approach, the recombinant proteins recovered can be cleaved from the oil bodies as a first purification step, for instance using an endoprotease. Depending on the recombinant protein and the oil-body protein used but also on the type of protein fusion, which may be an end-to-end connection or a protein linker, the cleavage step may be adapted. Further, the process of the invention may comprise the purification of the recombinant protein according to conventional methods.

Examples of plants which may be used are listed: Arabidopsis (Arabidopsis thaliana), Camelina (Camelina spp), Rapeseed (Brassica spp.), Soybean (Glycine max), Sunflower (Helianthus annuus), oil palm (Elaeis guineeis), cottonseed (Gossypium spp.), groundnut (Arachis hypogaea), coconut (Cocus nucifera), castor (Ricinus communis), Safflower (Carthamus tinctorius), mustard (Brassica spp. and Sinapis alba), coriander (Coriandrum Sativum), Squash (Cucurbita maxima), linseed/flax (Linum usitatissimum), Brazil nut (Bertholletia excelsa), jojoba (Simmondsia chinensis) and maize (Zea mays). However, other plants may be suitable for the present invention.

A list of sequences is provided according to Table 1.

TABLE 1

| Sequence | |
|---|---|
| number | Title |
| SEQ ID NO 1 | Transcript sequence of RdR2 (AT4G11130.1)/ |
| | Chr4:6780522 . . . 6784440 forward |
| SEQ ID NO 2 | Transcript sequence of RdR2 (LOC104707406) NCBI |
| | Reference Sequence: XM_010423753.2 |
| SEQ ID NO 3 | Transcript sequence of RdR2-like (LOC104737110) |
| | NCBI Reference Sequence: XM_010457214.2 |
| SEQ ID NO 4 | Transcript sequence of RdR2-like 2 |
| | (LOC104719267) NCBI |
| | Reference Sequence: XM_019230033.1 |
| SEQ ID NO 5 | Transcript sequence of RdR6 (AT3G49500.1)/ |
| | Chr3:18349161 . . . 18353624 reverse |
| SEQ ID NO 6 | Transcript sequence of RdR6 (LOC104711463) NCBI |
| | Reference Sequence: XM_019229277.1 |
| SEQ ID NO 7 | Transcript sequence of RdR6 |
| | variant (LOC104791175) NCBI |
| | Reference Sequence: XM_019246751.1 |
| SEQ ID NO 8 | Transcript variant of RdR6-like (LOC104780770) NCBI |
| | Reference Sequence: XM_010505306.2 |
| SEQ ID NO 9 | Binary vector pLX-CaMV, complete sequence |
| | MH200606 REGION: 260 . . . 1219 |
| SEQ ID NO 10 | Cloning vector Cloning vector |
| | pG2RNAi2, complete sequence/ |
| | KT954097 REGION: 2921 . . . 3429 |
| SEQ ID NO 11 | Example of oleosin fused to a part of the receptor binding |
| | domain of the Spike protein of SARS-CoV-2 by the |
| | intermediary of the prochymosin sequence |

Seq. 1 is an example of transcript sequence of RdR2 in Arabidopsis thaliana; Seq. 2 is an example of transcript sequence of RdR2 in Camelina sativa; Seq. 3 is an example of transcript sequence of RdR2-like in Camelina sativa; Seq. 4 is another example of transcript sequence of RdR2-like in Camelina sativa; Seq. 5 is an example of transcript sequence of RdR6 in Arabidopsis thaliana; Seq. 6 is an example of transcript sequence of RdR6 in Camelina sativa; Seq. 7 is an example of variant transcript sequence of RdR6 in Camelina sativa; Seq. 8 is an example of another variant transcript sequence of RdR6-like in Camelina sativa; Seq. 10 is an example of a strong promoter being a Double 35S CaMV promoter; Seq. 11 is an example of a strong promoter being a CSMV promoter; Seq. 11 is an example of an amino acids sequence an oleosin fused to a part of the receptor binding domain of the Spike protein of SARS-COV-2.

Advantageously, the strong promoter comprises or consists of the sequence of a Double 35S CaMV promoter set forth in SEQ. 9 or a variant thereof with at least 70% identity thereto, preferably 90% identity. Such promoters may be used with any embodiment of the present invention.

Advantageously, the strong promoter comprises or consists of the sequence of a CSMV promoter set forth in SEQ. 10 or a variant thereof with at least 90% identity thereto. Such promoter may be used with any embodiment of the present invention.

Advantageously, the coding sequence of a recombinant protein being the receptor binding domain of the Spike protein of SARS-COV-2 fused to an oleosin comprises a sequence having at least 90% sequence identity to SEQ. 11 (amino acids) or SEQ. 12 (DNA). Such sequence may be used for the nucleic acid construct comprising a coding sequence of a recombinant protein in a reading frame with an oil-body protein coding sequence of any embodiment of the present invention. A fusion protein which is enzymatically active and resides on the oil bodies may be used directly. The fusion protein may be stable in dry seeds for long periods, such as 2 to 4 years and when extracted has a half-life of 3 to 4 weeks on oil bodies.

The genetically engineered plant or part thereof of the present invention and the methods and process of the present invention allow the production of recombinant proteins with oil bodies and recombinant proteins purified, in a manner which is extremely inexpensive, with plants which may allow switching to open-field production, offering a novel route to the manufacture of recombinant proteins.

The present invention is directed to genetically engineered plants which have been modified locally so as to obtain a transient expression of a recombinant protein. The present invention is also directed to the genetically engineered plants which have been obtained from the seeds of a previous genetically engineered plant according to the invention and which allow a permanent expression of said recombinant protein.

The examples described above are given as illustrations of embodiments of the invention. They do not in any way limit the scope of the invention which is defined by the following claims.

1. A genetically engineered plant or part thereof adapted for the production of recombinant proteins comprising:
at least one inactivated gene involved in the Transcriptional Gene Silencing (TGS) and/or Post Transcriptional Gene Silencing (PTGS) mechanism corresponding to:
(1) at least one knocked-out gene involved in the TGS and/or PTGS mechanism; and/or
(2) at least one nucleic acid RNA interference construct targeting the RNA molecules expressed from the at least one gene involved in the TGS and/or PTGS mechanism;
at least one nucleic acid construct comprising a strong promoter coupled to a polynucleotide sequence comprising a coding sequence of a recombinant protein;
wherein (i) the coding sequence of the recombinant protein is linked in a reading frame to an oil body protein coding sequence; and/or (ii) the at least one inactivated gene involved in the TGS and/or PTGS mechanism includes at least one inactivated gene involved in the TGS mechanism and at least one inactivated gene involved in the PTGS mechanism.

2. The genetically engineered plant or part thereof according to claim 1, wherein the at least one inactivated gene involved in the TGS or PTGS mechanism is chosen among the Dicer-like (DCL) family, the Suppressor of Gene Silencing (SGS) family, the Argonaute (AGO) family, the RNA dependent RNA polymerase (RdR) family and combinations thereof.

3. The genetically engineered plant or part thereof according to claim 1, wherein the at least one inactivated gene involved in the TGS and/or PTGS mechanism includes the RdR2 gene and the RdR6 gene.

4. The genetically engineered plant or part thereof according to claim 1, the oil body protein coding sequence being the coding sequence of an oil body protein selected among oleosin, caleosin, steroleosin, perilipin or caveolin.

5. The genetically engineered plant or part thereof according to claim 1, wherein the coding sequence of a recombinant protein is selected among the coding sequence of a protein selected among antibodies, growth factors and antigens.

6. The genetically engineered plant or part thereof according to claim 1, wherein the coding sequence of a recombinant protein is the coding sequence of a protein of the SARS-COV-2 virus.

7. The genetically engineered plant or part thereof according to claim 1, the plant being a genetically engineered oleaginous plant or part thereof, preferably selected among the following genus: Arachis, Camelina, Theobroma, Brassica, Gossypium, Olea, Ricinus, Glycine, Helianthus, Elaeis, Carthamus and Linum.

8. The genetically engineered plant or part thereof according to claim 1, being a genetically engineered Nicotiana spp and preferably Nicotiana benthamiana or Camelina sativa or part thereof.

9. The genetically engineered plant or part thereof according to claim 1, the said part being at least one part selected among: the seeds, the pollens, the flowers, the roots, the stems, the mega- or micro-spores, the embryos and the vegetative organs of non-flowering plants.

10. The genetically engineered plant or part thereof according to claim 1, the strong promoter being a viral promoter.

11. A nucleic acid construct comprising a strong promoter coupled to a polynucleotide sequence comprising a coding sequence of a recombinant protein linked in a reading frame to an oil body protein coding sequence.

12. A process for producing a recombinant protein in a genetically engineered plant or part thereof, comprising the steps of:
inactivating at least one gene involved in the TGS and/or PTGS mechanism in a plant or part thereof by:
(1) generating at least one genetic alteration in the coding sequence of the at least one gene involved in the TGS and/or PTGS mechanism, such generating step being performed by using a method selected among: CRISPR-Cas 9, TALENs, Meganuclease, TILLING, Zinc finger nuclease, synthetic RNAi, physical or chemical mutagenesis and combinations thereof; and/or
(2) inserting in the plant at least one nucleic acid RNA interference construct targeting the RNA molecules expressed from the at least one gene involved in the TGS and/or PTGS mechanism;
inserting in the plant at least one nucleic acid construct comprising a strong promoter coupled to a polynucleotide sequence comprising a coding sequence of a recombinant protein,
wherein (i) the coding sequence of the recombinant protein is linked in a reading frame to an oil body protein coding sequence; and/or (ii) the step of inactivating at least one gene includes the inactivation of at least one gene involved in the TGS mechanism and at least one gene involved in the PTGS mechanism,
wherein the step of inserting is performed by using a method selected among: CRISPR-Cas 9, TALENs, Meganuclease, Zinc finger nuclease with particle gun transformation and Agrobacterium tumefaciens,
wherein the step of inactivating may be performed after the step of inserting, and optionally recovering the recombinant protein and the oil body protein and optionally purifying the recombinant protein recovered.

13. A method for obtaining a genetically engineered plant or part thereof as in claim 1, the method comprising:
inactivating at least one gene involved in the TGS and/or PTGS mechanism in a plant or part thereof by:
(1) generating at least one genetic alteration in the coding sequence of one or more gene involved in the TGS and/or PTGS mechanism, such generating step being performed by using a method selected among: CRISPR-Cas 9, TALENs, Meganuclease, TILLING and Zinc finger nuclease, synthetic RNAi, physical or chemical mutagenesis and combinations thereof; and/or
(2) implementing at least one nucleic acid RNA interference construct targeting the RNA molecules expressed from the TGS and/or PTGS mechanism,
inserting in the plant at least one nucleic acid construct comprising a strong promoter coupled to a nucleic acid sequence comprising a coding sequence of a recombinant protein,
wherein (i) the coding sequence of the recombinant protein is linked in a reading frame to an oil body protein coding sequence; and/or (ii) the step of inactivating at least one gene includes the inactivation of at least one gene involved in the TGS mechanism and at least one gene involved in the PTGS mechanism,
wherein the step of inserting is performed by using a method selected among: CRISPR-Cas 9, TALENs, Meganuclease, Zinc finger nuclease with particle gun transformation and Agrobacterium tumefaciens, and
wherein the step of inactivating may be performed after the step of inserting.

14. A method as in claim 13, wherein the method further comprises:
collecting the seeds of the genetically engineered plant with at least one inactivated gene involved in the TGS and/or PTGS mechanism and the at least one nucleic acid construct inserted;
growing new plants from the seeds and/or plant tissues;
selecting the heterozygotes or homozygotes new-grown plants which have at least one inactivated gene involved in the TGS and/or PTGS mechanism and the nucleic acid construct.

15. A fusion protein comprising a recombinant protein and an oil-body protein produced by, or obtained from, the genetically engineered plant or part thereof according claim 1.

16. A fusion protein comprising a recombinant protein and an oil-body protein produced by, or obtained from, the genetically engineered plant or part thereof obtained from the method of claim 13.

17. A fusion protein comprising a recombinant protein and an oil-body protein produced by, or obtained from, the heterozygotes or homozygotes new-grown plants selected of claim 14.


SEQUENCE LISTING (11 sequences)

SEQ ID NO: 1 - DNA, 3402 bp, Arabidopsis thaliana
atggtgtcagagacgacgacgaaccgatcaacggttaaaatctcaaacgttcctcaaaccatagtcgccgacgaactcctccggttcttagaactccacctcggcgaagatactgtattcgcactcgaaatcccaacaactcgtgacaattggaagccaagagacttcgctcgagtacaattcactaccctcgaagtcaaatctcgagcccagcttctctcttctcaaagcaagctccttttcaaaacccataacctaagactctctgaagcttatgatgatatcatccctcgtcctgtagatccaaggaaaaggctagacgacatcgttttgactgttgggtttcctgagtctgatgaaaaaaggttttgtgccctagaaaaatgggatggtgtgaggtgttggattttgactgagaagagaagagttgagttttgggtttgggaaagtggtgattgttataagattgaagttaggtttgaggatattattgaaactcttagttgttgtgttaatggtgacgcttctgagattgatgcatttcttctcaagctgaaatatggaccgaaggtatttaaaagagtaacagttcacatagccacaaagtttaagtctgatcggtacagattctgcaaggaggattttgatttcatgtggattcggacaactgatttctctggttcgaaatcaattggtacatcaacttgcttttgtttggaagtccacaatggctcaacgatgttagacattttctcgggcttaccttactaccgagaagacactttgagtctaacttatgtggatgggaagacctttgcttctgcagctcaaattgttcccctcttgaatgccgccattttggggttagagtttccatatgagatccttttccaactcaatgcgcttgttcatgcgcagaaaatcagtctttttgctgcgtcagatatggaactgatcaaaatccttcgtggtatgagtttggaaaccgcattggtgatcctcaagaaacttcaccagcaaagttctatatgttatgatcctgtcttctttgtcaagactcagatgcaatctgtggttaagaagatgaagcattcccctgcatcagcttataaaagattgaccgagcagaacatcatgagttgtcagagagcttacgttacaccctcaaagatctatctgttgggtccagagcttgagactgcaaattatgttgtgaagaactttgcagagcatgtctcggatttcatgagagttacttttgtggaagaagattggagcaagcttcccgcaaatgctctctctgtgaactccaaggaaggctattttgtgaagccctctagaaccaacatttataacagggtgttgtcaatccttggagaagggattaccgttgggcctaagaggtttgagttcttggctttttcagcgagccaactgcgaggaaattcggtctggatgtttgcttctaacgagaaagtaaaagcggaagatataagagaatggatgggttgtttccgtaaaatccgaagcatttccaaatgtgctgctaggatgggtcagttgttcagtgcttcgcggcaaacacttattgtccgtgcacaagatgtggagcagattcccgacatcgaagtgacaactgatggtgctgattactgcttctcggatggcattgggaaaatttcacttgcatttgccaagcaagttgcacagaagtgtggattgagtcatgtcccttctgcctttcaaattcgatacggtggctacaaaggtgtgattgctgttgaccgcagttccttccgaaagttgtctctgcgtgatagtatgcttaaatttgactcgaacaacaggatgctgaacgttaccaggtggacagagtcgatgccttgcttcttaaaccgggagatcatttgccttttgtcgacccttggaatagaagatgcaatgtttgaggcgatgcaagcggtgcatttatctatgcttgggaatatgcttgaagaccgcgatgcagcacttaatgttctgcagaaattgagcggagaaaattccaagaatttgctagttaagatgctgcttcaaggatatgcaccgagttcagaaccttacctctcaatgatgcttcgtgtgcaccacgagagccagctttctgaactaaagagcagatgcaggatacttgtaccgaaaggacggatcttgatcggttgcatggatgaaatgggtatcctggagtatggccaagtgtatgttcgtgtaaccctgactaaagcggaactgaaatctcgcgatcagagctactttcgcaagattgatgaggaaacatctgtggttattgggaaagtggtcgtgacgaaaaacccatgtcttcaccctggagatattagagttcttgatgctatatatgaggtccatttcgaagaaaagggatatctcgactgcatcatctttcctcagaagggagaaagaccacatccaaatgaatgttctggtggcgatctcgacggagaccagttttttgttagctgggatgagaagattatacctagcgaaatggaccctccaatggactatgctggaagcaggcctcgtctaatggatcatgacgtcacgttagaggaaatccacaaattttttgtggattatatgataagcgacacgcttggggtgatctcaactgcacatttggttcacgcagaccgtgaccctgaaaaagcccggagccagaagtgtctagagttagcaaatcttcactctagggctgttgattttgcaaaaactggagctccagctgagatgccttatgccttaaagcccagagagttccctgattttctggaacggtttgagaaaccaacatacatctctgagtctgtgtttgggaaactataccgtgctgtgaagagctctctagcacagagaaagccagaagctgagagcgaggacacggtagcttatgatgtgacacttgaagaagctggctttgagagctttatagagacagcgaaagcccatagagacatgtatggtgagaaactgacctcgttgatgatatactatggagctgctaacgaagaggagattctcacaggcattttgaaaaccaaggagatgtatctggcgagagataaccggaggtatggtgatatgaaggatagaattacgctttccgtgaaagatttgcataaagaggccatgggatggttcgagaaaagctgcgaggacgaacaacagaagaagaagctagcatcggcgtggtactatgtaacatacaatccaaaccatcgcgatgagaagttgacattcttgagtttcccatggatcgtaggtgacgttttgcttgatataaaggctgaaaatgcacagagacagagcgttgaagagaaaacaagtggacttgtatccatttga

SEQ ID NO: 2 - DNA, 3628 bp, Camelina sativa
ttttctgattctctgggaatcactcttcttcttcactccctctctctctctctctctctccgttcacttgctcttggggtcaatcatggtgtcagaggcgacgacggctacgaaccgaccaaccgttaaagtctcaaatgttcctcaaaccatagtcgccgacgaactcctccggttcctcgagctccacctcggcgaagacacagtgtacgctctcgaaatcccaacaactcgcgagaattggaagccacgggacttcgctagagtacaattcagtaccctcgaagtcaaatctcgagcccagcttctctcttcacaaagcaagctccttttcaaaacccataacctaagactctccgaagcttacgatgacatcatccctcgccctgtagatccacggagaaggctagaagacgtcgttttgactgttgggttccctgagtctggtgaagagaggttctgtgccctagaaaaatggggaggcgtgaggtgttggattctggaagagaagaggagacttgagttttgggtttgggaatgtggtgaatgttataagcttgaagttaggtttgaggatattgttgaaactcttggctgctgtgttaatggtgacgcttctgagattaatgcgtttcttctcaagctgaaatatggaccaaaggtttttaaaagagtgacaggacctcacatagccacaaagtttaaatctgatcggtaccgtttctgcaaggaggattttgatttcatgtgggttcgcacaactgatttctctggttcaaagtcaatcggtacatcaacttgcttttgtttggaggtagagaatggctccaagatgttagacattttctcgtgtttgccttactacagagaagacactttgagtctaaccactgtggatgggactgcgtttgcctctgcatgtcaaattgttcccctcttgaatgctgccagtctggggttagagtttccatatgagatcctctttcaactcaattcgctggttcacaatcagaaaatcagtctttttgctgcatcaaatatggaactgatcaacatccttcgtggtttgagcttggaaaccgcattggttatcctcaagaagcttcaccagcaaagctctatatgttatgatcctgtctcctttgtcaagactctgatgcaatccccggttaagaagatgaagcattcccctgcatcagctaatacaagactgaccgagcagaatatcatgagttgtcagagagcctacgttacaccctcaaagatttatctgttgggtccagagcttgagaaagctaactatgttgtgaagaactttgcagagcatgtctcagatttcatgagaattacttttgtggaagaagattggagcaagattccagcaaatgctctttctgtgaactccaaggaaggctattttgtgaagccctttagaaccaacatttataacagggtgttgtcaatccttggagaagggatcactgttgggcctaagaggtttgagttcttggctttttcagccagccaactgcgaggaaactcagtctggatgtttgcttctaacgagaaagttaaagcagaagatataagagaatggatgggttgtttccgtaaaatccgaagcatctccaaatgtgctgctaggatgggtcagttgttcagtgcttcacggcagacagttactgtccgtgcacaagatgtggagcagattcctgacattgaagtgactactgatggtgctgattattgcttctcggatggcatcgggaaaatttcgttggcatttgctaggcaagttgctcagaagtgtgggcttgattatgtcccttctgcatttcaaattcgatacggtggctacaaaggtgtgatcgccgttgaccgcagttccttcagaaagatgtctctgcgtgatagtatgctaaagtttgactcaaacaacaggatgcttaacgtaaccaggtggacagagtcgatgccttgcttcttaaaccgggagatcatttgtcttttgtcgactcttggaatagaagatgcagtgtttgaggccatgcaagcgatgcatttatctatgctggggaatatgcttgaagaccgcaatgcagcacttaatgtgctgcagaaattgagcggagaaaattccaagaatctgctagttaagatgctgctacaaggatacgcaccgagttcagaaccgtacctctcaatgatgcttcgtgtgcaccacgagagccaactttctgaactaaagagtaggtgcaggatacttgtaacgaaaggacggatcttgatcggttgcatggatgaaatgggtattctggagtatggccaagtgtatgttcgtgtaaccctgactgaagcggaactgaaatctcgtgaacagagttacttccgcaagattaatgaggaaacatctgtggtgattgggaaagtggtcgtgacgaaaaacccatgtcttcaccctggagatatcagagttcttgacgctatatatgaggtcagtttcgaagaaaagggatttgttgactgcatcatctttcctcagaagggagaaaggccacatcccaatgaatgttctggtggtgatctcgacggagaccagttttttgttagttgggatgagaagcttataccgagccaaatggaccctccaatggactacgctggaagcagacctcgtctaatggatcatgacgttaccctagaggaaatacacaaattttttgtggattatatgataagcgacacgctgggagtgatctcaactgcacatttggttcatgcagaccgtgaccctgaaaaagccaggagccagaagtgtctagagttagcacatcttcactctagggctgttgattttgcgaaaactggagctccagcggagatgccttatgccttaaagcccagagagttccctgatttcctggaacggttcgagaaaccaacatacatctctgagtctgtgtttgggaaactttaccgtgctgtgaaaagctcgttagcacagaggaagccagaagctgagagcgaggacacgatagcttatgattccacacttgaagaagctggctatgagagctttatagagacagccaaagcccacagagacatgtatgctgagaaactgacctcgttgatgatatactacggagctgtaaacgaagaagagatcctcacaggcattttgaaaactaaggagatgtatctggcgagagataaccggaggtatggtgatatgaaggatagaatcaccttgtctgtgaaggatttgcagagagaggccttgggatggttcgataaaagctgcgaggagaaacaagagaagaagaagctagcttcagcttggtattacgtaacatacaacccgagccatgtcgatgagaagttgaaattcctgagttttccgtggatcgttggtgacatattgctagagattaaggctgaaaatgcacagagtcagagcgttgaagaccaaacaaatggacttgtatccatttgattaccctatccctatctatctatcactcgttgctgtagtataattgctagattaagacaattgagaccagaacagaacgtcgcttgtatatgcgtcactcaaaattatcaaatactaatgactaattaatta

SEQ ID NO: 3 - DNA, 3377 bp, Camelina sativa
caacggttaaagtctccaatgttcctcaaaccatagtcgccgacgaactcctccgcttcctcgagcttcacctcggcgaagacacagtgttcgctctcgaaatcccaacaactcgcgagaattggaagccacgggacttcgctcgagtacaattcagtaccctcgaagtcaaatctcgagcccagcttctctcttcacaaagcaagctccttttcaaaacccataacctaagactctccgaagcttacgatgacatcatacctcgccctgtagatccacggagaaggctagaagacgtcgttttgactgttgggtttcctgagtctggtgaagagaggttctgtgccctagaaaaatggggaggcgtgaggtgttggattctggaagagaagaggagacttgagttttgggtttgggaatgtggtgaatgttataagattgaagttaggtttgaggatattgttgaaactcttggctgcgttgttaatggtgacgcctctgagattaatgcgtttcttctgaggctgaaatatggaccaaaggtatttaaaagagtaacaggacctcacatagccgcaaagtttaaatctgatcggtaccgtttctgcaaggatgattttgatttcatgtgggttcgcacaactgatttctctggttcaaagtcaatcggtacatcaacttgcttttttttggaggtagagaatggctccaagatgttagacattttctcgtggttgccttactacagagaaggcactttgagtctaaccactgtggatgggaaagcgtttgcctcttcatgtcaaattgttcccctcttgaatgctgccaatctggggttagagtttccatatgagatccttttccaactcaattcgctggttcacaataaaaaaatcagtctttttgctgcatcaaatatggaactgatcaacatccttcgtgggttgagattggaaaccgcattggttatcctcaagaaacttcaccagcaaagctctatatgttatgatcctgtctcctttgtcaagactcagatgcaatccccggttaagaagatgaagcattcccctgcatcagctaatacaagactgaccgagcagaatatcatgagttgtcagagagcctacgttacaccctcaaagatttatctgttgggtccagagcttgagaaagctaactatgttgtgaagaactttgcagagcatgtctcagatttcatgagaattacttttgtggaagaagattggagcaagattccagcaaatgctctttctgtgaactccaaggaaggctattttgtgaagccctttagaaccaacatttataacagggtgttgtcaatccttggagaagggatcactgttgggcctaagaggtttgagttcttggctttttcagccagccaactgcgaggaaactcagtctggatgtttgcttctaacgagaaagttaaagcagaagatataagagaatggatgggttgtttccgtaaaatccgaagcatctccaaatgtgctgctaggatgggtcagttgttcagtgcttcacggcagacagttactgtccgtgcacaagatgtggagcagattcctgacattgaagtgactactgatggtgctgattactgcttctcggatggcattgggaaaatttcattggcatttgctaggcaagttgctcagaagtgtgggcttgattatgtcccttctgcatttcaaattcgatacggtggctacaaaggtgtgatcgccgttgaccgcagttccttcagaaagatgtctctgcgtgatagtatgctaaagtttgactcaaacaacaggatgcttaacgtaaccaggtggacagagtcgatgccttgcttcctaaaccgggagatcatttgccttttgtcgactcttggaatagaagatgaagtgtttgaggcgatgcaagcgttgcatttatctatgctggggaacatgcttgaagaccgcaatgcagcacttaatgtgctgcagaaattgagcggagaaaattccaagaatctgctagttaagatgctgctacaaggatacgcaccgagctcagaaccgtacctctcaatgatgcttcgtgtacaccacgagagccagctttctgaactaaagagtaggtgcaggatacttgtaccgaaaggacggatcttgatcggttgcatggatgaaatgggtatcctggagtatggccaagtgtatgttcgtgtaaccctgactgaagcggaactgaaatctcgtgaacagagttacttccgcaaggttaatgaggaaacatctgtggtgattgggaaagtagtcgttacgaaaaacccatgtcttcaccctggagatatcagagttcttgacgctatatatgaggtcagtttcgaagaaaagggatttgtcgactgcatcatctttcctcagaagggagaaaggccacatcccaatgaatgttctggtggtgatctcgacggagaccagttttttgttagttgggatgagaagcttataccaagccaaatggaccctccaatggactacgctgctagcagacctcgtataatggatcatgatgtcaccctagaggaaatccacaaattttttgtggactatatgataagcgacacgctgggagtgatctcaactgcacatttggttcatgcagaccgcgaccctgaaaaagccaggagccagaaatgtctagagttagcgcatcttcactctagggctgttgattttgccaaaactggagctccagcggagatgccttacgccttaaagcccagagagttccctgatttcctggaacggttcgagaaaccaacatacatctctgagtctgtgtttgggaaactttaccgtgctgtgaaaagctcgttagcacaaaggaagccagaagctgagagcgaggacacgatagcttatgattccacacttgaagaagctggctatgagagctttatagagacagccaaaacccacagagacatgtatgctgagaaactgacctcgttgatgatatactacggagctgtaaacgaagaagagatcctcacaggcattttgaaaactaaggagatgtatctggcgagagataaccggaggtatggtgatatgaaggatagaatcaccttgtctgtgaaggatttgcagagagaggccttgggatggttcgagaaaagctgcgaggagaaacaagagaagaagaagctagcctcggcttggtactacgtaacatacaacccgagccaggtcgatgagaagttgaaattcctgagttttccgtggatcgttggtgacattttgctagagattaaggctgaaaatgcacagagtcagagcggtgaagaccaaacaaatggacttgtatccgtttga

SEQ ID NO: 4 - DNA, 3111 bp, Camelina sativa
cctgtagatccacggagaaggctagaagacgtcgttttgactgttgggttccctgagtctggtgaagagaggttctgtgccctagaaaaatggggaggcgtgaggtgttggattctggaagagaagaggagacttgagttttgggtttgggaatgtggtgaatgttataagcttgaagttaggtttgaggatattgttgaaactcttggctgctgtgttaatggtgacgcctctgagattaatgcgtttcttctcaagctgaaatatggatcgaaggtttttaaaagagtgacaggacctcacatagccacaaagtttaaatctgatcggtaccgtttctgcaaggaggattttgatttcatgtgggttcgcacaactgatttctctggttcaaagtcaatcggtatatcaacttgcttttgtttggaggtagagaatggctcaaagatgttagacattttctcaagcttgccttactacagagaagacacgttgagtctaaccactgtggatgggaaagtgtttgcctctgcatgtcaaattgttcccctcttgaatgctgccaatcttgggttagagtttccatatgagatcctttaccaactcaattcgcttgttcacaatcagaaaatcagtatttttgctgcgtcaaatatggaactgattaacatccttcgtggtttgagcttggaaaccgcattggtgatcctcaagaaacttcaccagcaaagctctatatgttatgatcctgtctcctttgtcaagactcagatgcaatccccggttaagaagatgaagcattcccctgcatcagctaatacaagattgaccgagcagaatatcatgagttgtcagagagcctacgttacaccctcaaagatttatctgttgggtccagagcttgagaaagctaactatgttgtgaagaactttgcagagcatgtctcagatttcatgagaattacttttgtggaagaagattggagcaagattccagcaaatgctttttctgtgaactccaaggaaggctattttgtgaagccctttagaaccaacatttataacagggtgttgtcaatccttggagaagggatcactgttgggcctaagaggtttgagttcttggctttttcagccagccaactgcgaggaaactcagtatggatgtttgcgtctaacgagaaagttaaagcagaagatataagagaatggatgggttgtttccgtaaaatccgaagcatatcaaaatgtgctgctcggatgggtcagttgttcagtgcttcacggcagacagttactgtccgtgcacaagatgtggagcagattcctgacattgaagtgactactgatggtgctgattactgcttctcggatggcatagggaaaatttcattggcatttgctaagcaagttgctcagaagtgtggactggattatgtcccttctgcatttcaaattcgatacggtggctacaaaggtgtgatcgccgttgaccgcagttccttcagaaagatgtctctgcgtgatagtatgctaaagtttgactcaaacaacaggatgcttaacgtaaccaggtggacagagtcgatgccttgcttcttaaaccgagagatcatttgccttttgtcgactcttggaattgaagatgaagtgtttgaggcgatgcaagctatgcatttatctatgctagggaatatgcttgaagaccgcaatgcagcacttaatgtgctgcagaaattgagcggagaaaattccaagaatctgctagttaagatgctgctacaaggatatgcaccgagttcagaaccgtacctctcaatgatgcttcgtgtacaccacgagagccaactttctgaactaaagagtaggtgcaggatacttgtaccgaaaggacggatcttgatcggttgcatggatgaaatgggtatcctggattatggccaagtgtatgttcgtgtaaccctgactgaagcggaactgaaatctcgtgaacagagctacttccacaagattaatgaggaaacatctgtggtgattgggaaagtggtcgttacgaaaaacccatgtcttcaccctggagatatcagagtacttgacgctatatatgaggtcagtttcgaagaaaagggatttgtcgactgcatcatctttcctcagaagggagaaaggccacatccaaatgaatgttctggtggtgatctcgacggagaccagttttttgttagttgggatgagaagcttataccaaaccaaatggaccctccaatggactacgctgcaagcagacctcgtctaatggatcatgacgttaccctagaggaaatacacaatttttttgtggattatatgataagcgacgcgcttgggatgatctcaactgcacatttggttcatgcagaccgtgacccagaaaaagccaggagccagaagtgtctagagctagcacatcttcactctagggctgttgattttgccaaaactggagctccggctgagatgccttatgccttaaagcctagagagttccctgatttcttggaacggttcgagaaaccaacatacatctctgagtctgtgtttgggaaactttaccgtgctgtgaaaagctcgttagcacagagaaagccagaagctgagagcgaggacacgatagcttatgattcgacacttgaagaagctggctttgagagctttatagagacagccaaagcccatagagacatgtatgctgagaaactgacctcgttgatgatatactacggagctgttaacgaagaagagatcctcacaggcattttgaaaaccaaggaaatgtatctggctagggataaccggaggtatggtgatatgaaggatagaatcacgttgtctgtgaaggatttgcagagagaggccatgggatggttcgacaaaagctgcgtggagaaacaagagaagaagaagctagcttcggcttggtactacgtaacatacagcccgagccaggtcgatgagaagttgagattcttgagttttccgtggatcgttggtgacattttgctagagatcaaggctgaaaatgcacagagacagagcgttgaagaccaaacaagtggacttgtatccatttga

SEQ ID NO: 5 - DNA, 3591 bp, Arabidopsis thaliana
atggggtcagagggaaatatgaagaagtcggttgtgacacaagtaagcattggtggatttggggaatccactactgcaaaacaactcacagattaccttgaagatgaagtaggaattgtttggcgttgcagattaaaaacttcgtggactcctcctgggtcttatcctaattttgaaatcgcagacacttcaaatatccccagtattgatgaatataagaaagtggagcctcatgcctttgtccatttcgcagtttttgaatcagctggtcgtgccatggatgctgcagggcaatgcaacctcatcttggatggccagccgttaaaagtcagtttgggtcctaagaatccatattcccttaatcaaagaagaagaacgactgtaccttataagttggctggtattacacttgagattgggacattggtttctcgggatgatttttttgtttcttggagagctgaaggggttgatttcctggtggatccttttgacaacacgtgcaagttttgtttcagaaagagcactgccttctctttcaaggatgcagtgatgcatgctgtgatcaattgtgattataagttggagttattggtgagagatatacaaacagtcaggcagtataaaaccttgcatggctttgtgcttattttgcagctggcttcttcaccccgtgtctggtacagaactgctgatgatgatatttacgatactgtccctggcgatctcttggatgatgatgatccttggatccgtaccactgattttactcaagttggggcaattggtcgatgtcattcatatcgagtgctcatatctccgcggtatgaaaataaactgagaacagccttagattattttaggatgcggagggtgcaagaggaacgtgtgaggtggcctccccgtatccgtaatgagccttgttttggggagcctgtgtcagatcatttcttctgcattcatcacaaggaaggaatctcttttgagatcatgtttctagtaaattcggtattgcacaggggtgtctttaaccagtttcagttgactgagcgattctttgatcttctaagaaaccaacccaaggacgtcaatatagcttctctcaagcatctctgtacctataaacgaccagtttttgatgcgtacaagaggttgaagcttgttcaggaatggattcagaaaaatccaaagcttttagggagtcatgaacaatctgaggatatctctgagatcagaagactagtaattaccccaaccagagcctattgcctacccccagaagttgagctctccaacagggtactcaggagatacaaagctgttgctgaaagatttttgcgggtaactttcatggatgaaagtatgcagaccataaattcgaatgttctctcttactttgttgctcctattgtgaaggatttgacatcaagttctttctcccagaagacctacgtttttaaaagagtgaagagcatattaaccgatgggtttaaactatgtggtagaaaatacagttttctagcattctcagccaatcaactgagagaccgctctgcatggttctttgctgaagacgggaaaacacgtgtgtcagatataaaaacatggatggggaagttcaaagacaagaatgtggcaaaatgtgctgctaggatgggcctgtgcttctcctccacatatgccactgtagatgtcatgcctcacgaggttgacactgaggttccagatattgagagaaatgggtatgttttctctgacggaattggtacaatcacacctgacctcgctgacgaagtaatggagaaacttaagttggatgtgcactacagcccttgtgcttatcagatacgttacgcaggtttcaaaggggttgttgctcgttggccatcaaaaagtgatggaatcaggctagcccttcgagacagtatgaagaagttcttttccaaacatacgatcttggagatctgttcctggacgaggtttcaacctgggttcttaaatcggcagataattacccttctatccgtactaggtgttccggatgaaatattctgggatatgcaggaatccatgctctataaactgaaccgcatccttgatgatacagatgtggcatttgaagttctcacggcatcatgtgctgaacagggaaacactgcagctatcatgcttagtgcaggtttcaaaccaaaaaccgagccgcatctacgcgggatgttgtcttcagtcagaattgcacaactctggggtctcagagaaaaatctcgtatttttgttacttcaggaaggtggctaatgggttgcctagacgaagcagggatacttgaacatggccaatgctttattcaagtctctaaaccgtctatagaaaattgtttctccaaacatgggtctcgttttaaggagacaaagacagatctggaagtagttaaaggctatgtagccattgctaagaatccttgtcttcacccaggggatgtaaggattttagaagctgttgatgtaccccagctgcatcacatgtatgactgccttattttccctcagaaaggtgataggccgcatacaaacgaagcttctggcagtgaccttgacggggacctgtactttgtggcttgggatcagaaactcatccctcccaacaggaaaagctatccggccatgcattatgatgcagctgaagagaagagtttaggccgtgctgtcaaccaccaggacataatcgatttctttgcaagaaacttggcgaatgagcagttgggcacaatttgcaatgcacacgtcgttcatgctgatagaagtgagtatggagccatggacgaagaatgtttgctactggcagaactagctgccactgcagttgatttcccaaagacagggaaaattgtgtcaatgcccttccacctaaaaccaaaactctacccagatttcatgggaaaagaagactaccaaacttacaagtcgaacaaaatcttgggtcggctttacagacgggtaaaagaggtttatgatgaagatgcagaagcttcctcagaagaaagcacagacccaagtgccatcccttatgacgctgttcttgaaataccgggatttgaagatttgatccctgaggcatggggtcacaaatgtttgtacgacgggcaactcattggtcttcttgggcaatacaaggtgcagaaagaggaagagattgtgacgggtcacatctggtccatgcccaaatacacaagcaagaaacaaggcgaactgaaagaaagactgaagcattcttataattcccttaaaaaggagttcaggaaagtatttgaggaaacaatccctgaccatgaaaatctcagcgaggaggaaaaaaacatcttgtatgagaagaaggcttcagcttggtatcatgtaacttaccatcccgagtgggtgaagaagtctttagagctgcaagatccagatgagtccagtcatgcggcgatgctgagttttgcttggattgcagctgattatcttgcaagaatcaaaatccggtcacgggaaatgggaagtatcgactcagccaagcctgttgattctttggctaagtttcttgctcagcgtctctaa

SEQ ID NO: 6 - DNA, 3965 bp, Camelina sativa
cgtgggaggaagtcaagaataataatgacgcatggtttcactttcagctatggcgatttctccttcctaatttagcctctactcacttactaatcttctctccggtctccgcgttgctatctgtttcgtcttcagtatcgatttcgttcactgtggcttcacggagaatggggtcagagggaaacatgaagaactcggttgtgacacaagtaagcattggtggattcggtgagtccactactgcaaaacaacttactgattacctcgaagacgaagtaggacttgtttggcgatgcagattaaagaactcttggactccgcctgggtcttatcctaatttcgaaatcgcagacacttcaaaaatccccaaaatcgatgattacaagaaagtggagcctcatgcgtttgtccatttcgcacttcctgaatcaacagcacgtgctatggatgctgcagggcaatgcaagctcatcctcgatggccagcctttaaaagtcagcttggggcctaagaacccatatacacttaaccagagaagaagaacgactacaccttataagttgactggtgtttcgatcgagattgggactttggtttcccgggatgagtttcttgtttcttggagagctgaaggtgttgatttccttgtagatccttttgacaacacttgcaagttttgcttcaggaagagcactgcgttctctttcaaggacgcagtgatgcatgctgtgataaattgtgattataagttggagttgttggtgagagacataagaacggttaggcagtataaggatttgcatggctttgtgcttcttttgcagctggcttcgtcaccccgtgtctggtacagaactgcggatgatgacatttatgagactgttcctttcgatctcttggatgatgatgacccttggatccgtaccactgatttcactcaagttggggcgattggtcgatgtctttcgtatagagtgctcatatctcctcggtatgagaagaaattgacaacagctttagattatctaaggatgcggagggtgcaagaggaacgtgtgaggtggcctcctcggatccgtgatgagccttgttttggggagcctgtgtcggatcatttcttctgcattcatcacaaggaaggaatctcctttgagatcatgtttctagtgaactcggtattgcacagaggggtttttaaccagtttcagttgactgagcgtttctttgatcttctcagaaaccaacccagagatgtcaatatagcttctctcaagcatctctgtacctataaaaaaccagtttttgatgcgtacaagaggttgaagcttgttcaggaatggattcagaaaaatccaaagcttttagggagtcatgaacaatctgaggatatctctgagatcagaaggttagtgattaccccaaccagagcccattgcctacccccagaagttgagctctccaacagggtacttaggaaatacaaagctgttgctgaaagatttttgcgcgtaactttcatggatgaaagtatgcagaccataaattcgaatgttctctcttactttgttgctcctattgtgaaggatctgacatcaagctctttctctcagaagacttacgttttcaaaagagtgaagagcatattaaccgatgggtttaaactatgtggtagaaaatatagttttctagcattctcagccaatcaactgagagaccgctctgcttggttctttgctgaagacgggaaaacaagagtgtcagatataaaaacatggatggggaagtttaaagacaagaatgtggcaaaatgtgctgctaggatgggactgtgcttctcctccacatatgccactgtagatgtcatgcttcacgaggttgacaccgagcttccagacattgagagaaatgggtatgttttctctgatggaattggtacaatcacacctgacctcgctgacgaagtaatggagaaacttaggttggatgtgcactacaccccttgtgcttatcagatacgttacgcaggtttcaaaggagttgttgctcgttggccatcaaaaggtgatggaatccggctagcccttcgagacagtatgaagaagttcaattccaaacatactatcttggagatctgttcctggacgaggtttcaacctggattcttaaatcggcagataattactctcctttctgtactaggtgttccggatgaaatattctgggatatgcaggaatccatgctctgtaaactgaaccgcatccttgatgatacagatgttgcatttgaagttctcacagcatcatgtgctgaacagggaaacactgcagctatcatgctaagtgcaggtttcaaaccaaaaactgagccacatctacgtggaatgttgtcttcagtcagaattgcacaactctggggtctcagagaaaaatcacgtatttttgttacttcaggaaggtggctaatgggttgccttgacgaagcagggatacttgaacagggccaatgctttatccaagtctcaaaaccgtctatagaaaattgtttctccaaacatgggtctcgttttaaggagacaaagacagatctgcaagtagttaaaggctatgtagccattgccaagaatccttgtcttcacccaggggatgtaaggattttagaagctgttgatgtaccccagctgcatcacatgtatgactgccttattttccctcagaaaggtgataggccccatacaaacgaagcttctggcagtgaccttgacggggacctgtactttgtggcttgggatcagaaactcatccctcccagcaaaaaaagctttccggccatgcattatgatgcagctgaagagaagagtttaggccgtgctgtcaaccaccaggatataatagatttctttgctagaaacatggcaaatgagcatttgggtacaatttgcaatgcacacgtcgttcatgctgatagaagtgagtatggagccatggacgaagaatgtatgctgctggcagaattagctgccactgcagttgatttcccgaagacggggaaaattgtgtcaatgcccttccacctaaaaccaaaactctacccagatttcatgggaaaagaagactaccaaacttacaagtcgaacaaaatcttaggtcggctttacagacgtgtgaaagaagtgtatgatgaagatgcagaagcttcgtcagaagaaatcccagatccaagtgacatcccttatgacactgatctcgagttaccgggatttgcagacttgatccccgaggcatggggtcacaaatgttcttacgacgggcagctcattggtcttcttgggcaatacaaggtccagaaagaggaagagattgtgacgggccatatctggtcgatgccgaaatacgctagtaagaaacaaggggaattgaaagaaagattgaagcattcttataactccctcaaaaaggagttcagaaaagtattcgaggaaacaatcccggaccaggaaaatctcagcgatgaggaaaaaaacatcttgtatgagaagaaggcttcagcttggtatcatgtcacttaccatcccgagtgggtgaagaagtctttagagctgcaagatccagatgagtcgtctagtcatgcggtgatgctgagttttgcttggattgcagctgattatcttgcaagaatcaaaatccggtcacgggaaatgggaaatatcgactcagccaagcctgttgattctttggccaagtttcttgctcaacgtctctaaaaggtaatgttcgaagccgtaaggccactctgccatgtatctgtggaaattcacagatactcaatatatatatatataagctagttttgaatgtactactctggaaactttgtgtgtgatgaattcaggatttaaacagctcaactgtttgccgttgttttgtggttatgtattctactaaaatttcatttcaattttattaca

SEQ ID NO: 7 - DNA, 3981 bp, Camelina sativa
atgaagtaaaaaaaaaaaaaggtttcactttcagctatggcgatttcgccttcctaatttagcgcggcctactcactagtcttctctgcggtctccgcgttgcttctgtttcgtttccagtatcgatttcgttgattgaggacctcactgtagcagtgaattagggcttttgcgaaaaatggggtcagagggaaacatgaagaactcggttgtgacacaagtaagcattggtggatttggtgagtccactactgctaaacaacttacagattacctcgaagacgaagtaggacttgtttggcgatgcagattaaagaactcttggactccccctgggtcttaccctaatttcgaaatcgcagacacttcaaaaatccccaaaatcgatgattacaagaaagtggagcctcatgcgtttgtccatttcgctattcccgaatcaacagaacgtgccatggatgctgcagggcaatgcaagctcatcctcgatgggcagccgttaaaagtcagcttgggtcctaagaacccatatacacttaaccagagaagaagaacgactacaccttataagttgactggtgtttcgattgagattgggactttggtttcccgggatgagtttcttgtttcatggagagctgaaggggttgatttccttgtagatccttttgacaacacttgcaagttttgcttcaggaagagcactgcgttctctttcaaggacgcagtgatgcatgctgtgataaattgtgattataagttggagttgttggtgagagacataagaacagtaaggcagtataagaatttgcatggctttgtgcttcttttgcagctggcttcgtcaccccgtgtctggtacagaactgctgatgatgatatttacgagactgttccttttgatctcttggatgatgatgacccttggatccgtaccactgattttactcaagttggggcgatcggtcgatgtctttcttatcgagtgctcatatctcctcggtatgagaagaagttgacaacagcgttagattatctaaggatgcggagggtgcaagaggaacgtgtgaggtggcctcctcggatccgtgatgagccctcttttggggagcctgtgtcagatcatttcttctgcattcatcacaaggaaggaatctcctttgagatcatgttcctagtaaactcggtattgcacagaggggtttttaaccagtttcagttgactgagcgtttctttgatcttctcagaaaccaacccagagatgtcaatatagcttctctcaagcatctctgtacctataaaaaaccagtttttgatgcgtacaagaggttgaagcttgttcaggaatggattcagaaaaatccaaagcttttagggagtcatgaacaatctgaggatatctctgagatcagaaggttagttattaccccaaccagagcccattgcctacccccagaagttgagctctccaacagggttctcaggaaatacaaagctgttgctgaaagatttttgcgcgtaactttcatggatgaaagtatgcagactataaattctaatgttctctcttactttgttgctcctattgtgaaggatctgacatcaagctctttctctcagaagacttacgttttcaaaagagtgaagagcatattaaccgatgggtttaaactatgtggtagaaaatatagttttctagcattctccgccaatcaactgagagaccgctctgcatggttctttgctgaagatgggaaaacaagagtgtcagatataaaaacatggatggggaagttcaaagacaagaatgtggcaaaatgtgctgctaggatgggactgtgcttctcctccacatatgccactgtagatgtcatgcttcacgaggttgacaccgagcttccagacattgagagaaatgggtatgttttctctgatggaattggtacaatcacacctgacctcgctgacgaagtaatggagaaacttaggttggatgtgcactacaccccttgtgcttatcagatacgttacgcaggtttcaaaggagttgttgctcgttggccatcaaaaggtgatggaatccggctagctcttcgagacagtatgaagaagttcaattccaaacatactatcttggagatctgttcctggacgaggtttcaacctggattcttaaatcggcagataattactctcctttctgtactaggtgttccggatgaaatattctgggatatgcaggaatccatgctctgtaaactgaaccgcatccttgatgatacagatgttgcatttgaagttctcacagcatcatgtgctgaacagggaaacactgcagctatcatgctaagtgcaggtttcaaaccaaaaactgagccacatctacgtggaatgttgtcttcagtcagaattgcacaactctggggtctcagagaaaaatcacgtatttttgttacttcaggaaggtggctaatgggttgccttgacgaagcagggatacttgaacagggccaatgctttatccaagtctcaaaaccgtctatagaaaattgtttctccaaacatgggtctcgttttaaggagacaaagacagatctgcaagtagttaaaggctatgtagccattgccaagaatccttgtcttcacccaggggatgtaaggattttagaagctgttgatgtaccccagctgcatcacatgtatgactgccttattttccctcagaaaggtgataggccccatacaaacgaagcttctggcagtgaccttgacggggacttgtactttgtggcttgggatcagaaactcatccctcctagcaaaaaaagctttccggccatgcattatgatgcagctgaagagaagagtttaggccgtgctgtcaaccaccaggatataatagatttctttgcaagaaacatggcaaatgagcatttgggtacaatttgcaatgcacacgtcgttcatgctgatagaagtgagtatggagccatggacgaagaatgtatgctactggcagaattagctgccactgcagttgatttcccgaagacagggaaaattgtgtcaatgcccttccacctaaaaccaaaactctacccagatttcatggggaaagaagactaccaaacttacaagtcgaacaaaatcttgggtcgtctttacagaagggtaaaacaagtgtatgatgaagatgcagaagcttcttcagaagaaatcccagaccctagtgacatcccttatgacactgatcttgaaatacctggattcgcagagttgatccccgaggcatggggtcacaaatgttcttacgacgggcagctcattggtcttcttgggcaatataaggtccagaaagaggaagagattgtgacgggccatatctggtcgatgccgaaatacgctagtaagaaacaaggggaattgaaagaaagattgaagcattcttataactccctcaaaaaggagttcagaaaagtattcgaggaaacaatcccggaccaggaaaatctcagcgatgaggaaaaaaacatcttgtatgagaagaaggcttcagcttggtatcatgtcacttaccatcccgagtgggtgaagaagtctttagagctgcaagatccagatgagtcgtctagtcatgcggtgatgctgagttttgcttggattgcagctgattaccttgcaagaatcaaagtccggtcacgggaaatggaaaatatcgactcagccaagcctgttgattctttggccaagtttcttgctcaacgtctctaaaaggtaatgttcgaagccgtaaggccactctgccatgtatctgtggaaattcacagatactccaatatttgtaagctagttttgaatgtgctattctggaaactttgtgtgtgatgaattcaggatttaaacagctcaactgtttgccgttgtttgtggttatgtattttactaaactttcgtttcaattttattacatgggttgataa

SEQ ID NO: 8 - DNA, 3988 bp, Camelina sativa
tgggagagagagagtgggagaagtcaagaataatcatgacgcatggtttcactttcagctatggcgatttctccttcctaatttagcgcctactcacttactaatcttctctccggtctccgcgttgcttctgtttcgtcttcagtatcgattccgttcactgttggattagggctttacggagaatggggtcagagggaaacatgaagaactcggttgtgacacaagtaagcattggtggatttggtgagtccactactgcaaaacaacttacagattacctcgaagatgaagtaggacttgtttggcgatgcagattaaaaaactcttggactccccctgggtcttatcctaatttcgaaatcgcagacacttcaaaaatccccaaaatcaatgattacaagaaagtggagcctcatgcgtttgtccatttcgcgcttcctgaatcaacagcacgtgccatggatgctgcagggcaatgcaagctcatccttgatggccagcctttaaaggtcagcttggggcctaagaacccatatacacttaaccagagaagaagaacgactactccatataagttgactggtgtttcggtcgagattgggactttggtttcccgggatgagtttcttgtttcttggagagctgaaggggttgatttccttgtagatccttttgacaacacttgcaagttttgtttcaggaagagcactgcgttctctttcaaggatgcagtgatgcatgctgtgataaattgtgattataagttggagttgttggtgagagacataagaacagttaggcagtataagaatttgcatggctttgtgcttcttttgcagctggcttcttcaccccgtgtctggtacagaactgctgatgatgatatttacgagactgttcctttcgatctcttggatgatgatgacccttggatccgtaccactgattttacacaagttggggcgatcggtcgatgtctttcttatcgagtgctcatatctcctcggtatgagaagaagttgacaacggcattagattatctaaggatgcggagggtgcaagaggaacgtgtgaggtggcctcctcggatccgtgatgagccttgttttggggagcctgtgtcagatcatttcttctgcattcatcacaaggaaggaatctcctttgagatcatgttcctagtaaactcggtattgcacagaggggtttttaaccagtttcagttgactgagcgtttctttgatcttctcagaaaccaatccagagatgtcaatatagcctctctcaagcatctctgtacctataaaagaccagtttttgatgcgtacaagaggctgaagcttgttcaggaatggattcagaaaaatcccaagcttttagggagtcatgaacaatctgaggatatctctgagatcagaaggttagtgattaccccaaccagagcccattgcctacccccagaagttgagctctccaacagggtactcaggaaatacaaagctgttgcggaaagatttttgcgcgtaacattcatggatgaaagtatgcagaccataaattcgaatgttctctcttactttgtcgctcctattgtgaaggatctgacatcaagctctttctctcagaagacttacgttttcaaaagagtgaagagcatattaaccgatgggtttaaactatgtggtagaaaatatagttttctagcattctcagccaatcaactgagagaccgctctgcttggttctttgctgaagacgggaaaacaagagtgtcagatataaaaacatggatggggaagttcaaagacaagaatgtggcaaaatgtgctgctaggatgggactgtgcttctcctccacatatgccactgtagatgtcatgcttcacgaggttgacaccgagcttccagacattgagagaaatgggtatgttttctctgatggaattggtacaatcacacctgacctcgctgacgaagtaatggagaaacttaggttggatgtgcactacaccccttgtgcttatcagatacgttacgcaggtttcaaaggagttgttgctcgttggccatcaaaaggtgatggaatccggctagcccttcgagacagtatgaagaagttcaattccaaacatattatcttagagatctgttcctggacgaggtttcaacctggattcttgaaccggcagattattactctcctttctgtactaggtgttccggatgaaatattctgggatatgcaggaatccatgctctgtaaactgaaccgcatccttgatgataccgatgttgcatttgaagttctaacagcatcatgtgctgaacaggggaacactgcagctatcatgctaagtgcaggtttcaaaccaaaaactgagccacatctacgtggaatgctgtcttcagtcagaattgcacaactctggggtctcagagaaaaatctcgtatttttgttacttcaggaaggtggctgatgggttgccttgacgaagcagggatacttgaacagggacaatgctttatccaagtctcaaaaccgtctttagaaaattgtttctccaaacatgggtctcgttttaaggagacaaagacagatctgcaagtagttaaaggctatgtagccattgccaagaatccttgtcttcacccaggggatgtaaggattttagaagctgttgatgtaccccagctgcatcacatgtatgactgccttattttccctcagaaaggtgataggccccatacaaacgaagcttctggcagtgaccttgacggggacctgtactttgtggcttgggatcagaaactcatccctcccagcaaaaaaagctttccggccatgcattatgatgcagctgaagagaagagtttaggccgtgctgtcaaccaccaggatataatagatttctttgcaagaaacatggcaaatgagcatttgggtacaatttgcaatgcacacgtcgttcatgctgatagaagtgagtatggagccatggacgaagaatgtatgctactggcagaattagctgccactgcagttgatttcccgaagacagggaaaattgtgtcaatgcccttccacctaaaaccaaaactctacccagatttcatggggaaagaagactaccaaacttacaagtcgaacaaaatcttgggtcggctttacagaagggtaaaggaagtgtatgatgaagatgcagaagcttcctcagaagaaatcccagacccaagtgacatcccttatgacactgatcttgaaatacctggattcgcagatctgatccccgaggcatggggtcacaaatgttcttacgacgggcagctcattggtcttcttgggcaatataaggtccagaaagaggaagagattgtgacgggccatatctggtcgatgcctagatacgctagtaagaaacaaggggaattgaaagaaagattgaagcattcttataactccctcaaaaaggagttcagaaaagtattcgagggaacaatcccggaccaggaaaatctcagcgatgaggaaaaaaacatcttgtatgagaaaaaggcttcagcttggtatcatgtcacttaccatcccgagtgggtgaagaagtctttagagctgcaagatccagatgagtcgtctagtcatgcggtgatgctgagttttgcttggattgcagctgattatcttgcaagaatcaaaatccggtcacgggaaatgggaaatatcgactcagccaagcctgttgattctttggccaagtttcttgctcaacgtctctaaaaggtaatgttcgaagccgtaaggccactctgccatgtatctgtggaaattcacagatactccaatatatgtaagctagttttgaatgtgctattctggaaactttgtgtgtgatgaattcaggatttaaacagctcaactgtttgccgttgtttgtcgttatgtattttactaaactttcgtttcaattttattacatgggttgataa

SEQ ID NO: 9 - DNA, 960 bp, Cauliflower mosaic virus
ccagtacgcacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcacc

SEQ ID NO: 10 - DNA, 509 bp, Cassava vein mosaic virus
agaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaag

SEQ ID NO: 11 - DNA, 1161 bp, Artificial Sequence
  Oleosin sequence fused to the receptor binding domain of the Spike gene of SARS-CoV-2 by the intermediary of the prochymosin sequence
atggcggatacagctagaggaacccatcacgatatcatcggcagagaccagtacccgatgatgggccgagaccgagaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactactgaagattttttgcaaaagcaacaatatggaatttcttctaagttttggccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttgtga